(19) United States
(12) Patent Application Publication
(10) Pub. No.: US 2024/0304328 A1
(43) Pub. Date: Sep. 12, 2024

(54) METHOD OF DETECTING EARLY RECURRENCE OF LIVER CANCER

(71) Applicant: Yun Xiang Investment Consulting Co., Ltd., Zhudong Township (TW)

(72) Inventors: Chang-Yi LU, Hsinchu County (TW); Pey-Jey PENG, Hsinchu City (TW)

(21) Appl. No.: 18/582,577

(22) Filed: Feb. 20, 2024

Related U.S. Application Data
(60) Provisional application No. 63/451,215, filed on Mar. 9, 2023.

(51) Int. Cl. G16H 50/20; C12Q 1/6851; C12Q 1/6886

(52) U.S. Cl. CPC G16H 50/20; C12Q 1/6851; C12Q 1/6886; C12Q 2600/154

(57) ABSTRACT

A method of detecting early recurrence of liver has steps of performing identification of a plurality of differentially methylated genes in a computing system; performing qualitative measurement of methylation levels of the methylated genes and performing quantitative measurement of the methylation levels of the methylated genes in the computing system; performing construction of a methylation prediction model of the methylated genes based on the measured methylation levels of the methylated genes in the computing system; and performing detection of early recurrence of liver cancer with the methylation prediction model in the computing system.

### CROSS-REFERENCE TO RELATED APPLICATIONS

The present application claims the benefit of the U.S. provisional application Ser. No. 63/451,215, filed on Mar. 9, 2023, which is incorporated herein by reference in its entirety.

### TECHNICAL FIELD

The present invention relates to a method of detecting early recurrence (ER) of liver cancer by using differentially methylated genes as biomarkers and measuring methylation levels of these biomarkers. The differentially methylated genes may be selected from a group consisting of CRTC1, LTB4R2, MARCKS, LMO7, FOXL2, MEIS3, AGAP1, BDNF, NCAM1, PLA2G7, ZNF763, ZNF816A, KCNJ3, CDH11, HISTIH2BE, FGF13, GRIK3, NEUROD2, P2RY6, and PDE6B. More specifically, a methylation prediction model for early recurrence (ER) is developed for use in prognosis of liver cancer.

### BACKGROUND

Hepatocellular carcinoma (HCC) is known as one of the most fatal cancers worldwide, causing 830,000 deaths every year. The Barcelona Clinical Liver Cancer (BCLC) staging system, which is based on liver function and tumor status, is now widely adopted as a crucial staging system for predicting a patient's prognosis and guiding clinical treatment. Because of the advances in medical technology, several treatment strategies could be performed based on the stage of cancer, including hepatectomy, transplantation, ablation, embolization, targeted therapy, and immunotherapy. Among them, only hepatectomy and transplantation are considered curative treatments for small, early-stage HCC tumors. Although modern surgical techniques and proper perioperative management have reduced the mortality rate, the high recurrence rate after hepatic resection still disappoints long-term survival of surgical patients. Overall, the 5-year recurrence rate is as high as 70% [1]. HCC displays a unique recurrence pattern, described as a double-peaked incidence of recurrence, and can be divided into early and late recurrences by using 1 year as the cut-off [2]. In general, early recurrence (ER) is typically due to intrahepatic metastases of the primary tumors. Late recurrence, on the other hand, is caused by a “de novo” multicentric occurrence in the remnant liver [3]. Early recurrence is responsible for 60-70% of HCC recurrences and is highly associated with poor survival rates [2]. Early recurrence is also considered as one of the most important factors for prognosis after curative therapy of HCC.

There is an unmet medical need to identify the patients at high risk of early recurrence to improve the survival of this population through frequent surveillance, early diagnosis, and effective treatment. Postoperative adjuvant therapies, including repeat hepatectomy, local ablation, and transcatheter arterial chemoembolization (TACE) have been reported to improve the survival rates [4]-[6]. However, the accurate prediction of early recurrence is still challenging now. Currently, some clinicopathological information has been proposed and applied to evaluate the risk of recurrence after surgical resection, such as gender, albumin-bilirubin grade, tumor size, tumor number, microvascular invasion, and serum level of alpha-fetoprotein (AFP), alanine aminotransferase (ALT), and des-γ-carboxy-prothrombin (DCP) [7]-[10]. Besides, many efforts have been made to identify molecular markers for the prediction of recurrence and survival of HCC after curative treatment in recent years. Gene and microRNA (miRNA) signatures have been displayed to be potential prognostic biomarkers and correlated with recurrence and clinical outcomes [11]-[12]. However, up to now, there is still no widely adopted prediction tool for guiding postoperative management.

DNA methylation is one of the most important epigenetic regulations and plays a fundamental role in every stage of carcinogenesis by regulating cancer-related gene expression. Increasing evidence shows that DNA hypermethylation engages in the mechanism of repressing tumor suppressors and DNA repair genes, and DNA hypomethylation is responsible for the overexpression of oncogenes in many types of cancers, including HCC [13]. The use of these aberrantly methylated genes as cancer markers is emerging for the value of clinical applications from diagnosis, prognosis, therapy selection, and recurrence monitoring [14]. Methylation biomarkers possess several advantages over other types of biomarkers. For example, cytosine methylation is a covalent binding of a methyl group to genomic DNA. It is relatively stable compared with protein or RNA markers. Moreover, methylation biomarkers can be detected in many types of liquid biopsies, including blood, urine, saliva, and stool, offering a non-invasive way to monitor cancer progression [15]. Plenty of methods have been used in DNA methylation for quantitative and qualitative analysis. Especially, quantitative methylation-specific PCR (qMSP) provides a simple, fast, sensitive, and cost-effective tool that can be easily implemented into clinical applications.

The long-term survival of patients with hepatocellular carcinoma (HCC) who undergo surgery remains unsatisfactory due to the high recurrence rate after hepatic resection. There is an urgent need to identify patients who are at high risk of early recurrence to improve the overall survival of this group.

The following is a list of references that are occasionally cited in the above-mentioned description. The disclosures of these references are incorporated by reference herein in their entirety.
[1] Sherman M. N Engl J Med. Recurrence of hepatocellular carcinoma. 2008 Nov. 6; 359(19):2045-7. doi: 10.1056/NEJMe0807581. Epub 2008 Oct. 15.
[2] Dai W C, Cheung T T. Strategic overview on the best treatment option for intrahepatic hepatocellular carcinoma recurrence. Expert Rev Anticancer Ther. 2016 Oct.; 16(10):1063-72. doi: 10.1080/14737140.2016.1226136. Epub 2016 Aug. 27.
[3] Imamura H, Matsuyama Y, Tanaka E, Ohkubo T, Hasegawa K, Miyagawa S, Sugawara Y, Minagawa M, Takayama T, Kawasaki S, Makuuchi M. Risk factors contributing to early and late phase intrahepatic recurrence of hepatocellular carcinoma after hepatectomy. J Hepatol. 2003 Feb.; 38(2):200-7. doi: 10.1016/s0168-8278(02)00360-4. PMID: 12547409.
[4] Matsuda M, Fujii H, Kono H, Matsumoto Y. Surgical treatment of recurrent hepatocellular carcinoma based on the mode of recurrence: repeat hepatic resection or ablation are good choices for patients with recurrent multicentric cancer. J Hepatobiliary Pancreat Surg. 2001; 8(4):353-9. doi: 10.1007/s005340170008.
[5] Zhou Y, Sui C, Li B, Yin Z, Tan Y, Yang J, Liu Z. Repeat hepatectomy for recurrent hepatocellular carcinoma: a local experience and a systematic review. World J Surg Oncol. 2010 Jul. 1; 8:55. doi: 10.1186/1477-7819-8-55.
[6] Liu S, Guo L, Li H, Zhang B, Sun J, Zhou C, Zhou J, Fan J, Ye Q. Postoperative Adjuvant Trans-Arterial Chemoembolization for Patients with Hepatocellular Carcinoma and Portal Vein Tumor Thrombus. Ann Surg Oncol. 2018 Jul.; 25(7):2098-2104. doi: 10.1245/s10434-018-6438-1. Epub 2018 May 4.
[7] Chan A W H, Zhong J, Berhane S, Toyoda H, Cucchetti A, Shi K, Tada T, Chong C C N, Xiang B D, Li L Q, Lai P B S, Mazzaferro V, García-Fiñana M, Kudo M, Kumada T, Roayaie S, Johnson P J. Development of pre-and post-operative models to predict early recurrence of hepatocellular carcinoma after surgical resection. J Hepatol. 2018 Dec.; 69(6):1284-1293. doi: 10.1016/j.jhep.2018.08.027. Epub 2018 Sep. 18.
[8] Lim K C, Chow P K, Allen J C, Chia G S, Lim M, Cheow P C, Chung A Y, Ooi L L, Tan S B. Microvascular invasion is a better predictor of tumor recurrence and overall survival following surgical resection for hepatocellular carcinoma compared to the Milan criteria. Ann Surg. 2011 Jul.; 254(1):108-13. doi: 10.1097/SLA.0b013e31821ad884.
[9] Li T, Fan J, Qin L X, Zhou J, Sun H C, Qiu S J, Ye Q H, Wang L, Tang Z Y. Risk factors, prognosis, and management of early and late intrahepatic recurrence after resection of primary clear cell carcinoma of the liver. Ann Surg Oncol. 2011 July; 18(7): 1955-63. doi: 10.1245/s10434-010-1540-z. Epub 2011 Jan. 15.
[10] Wu J C, Huang Y H, Chau G Y, Su C W, Lai C R, Lee P C, Huo T I, Sheen I J, Lee S D, Lui W Y. Risk factors for early and late recurrence in hepatitis B-related hepatocellular carcinoma. J Hepatol. 2009 Nov.; 51(5):890-7. doi: 10.1016/j.jhep.2009.07.009. Epub 2009 Jul. 23.
[11] Wang Z, Zhang N, Lv J, Ma C, Gu J, Du Y, Qiu Y, Zhang Z, Li M, Jiang Y, Zhao J, Du H, Zhang Z, Lu W, Zhang Y. A Five-Gene Signature for Recurrence Prediction of Hepatocellular Carcinoma Patients. Biomed Res Int. 2020 Oct. 24; 2020:4037639. doi: 10.1155/2020/4037639. eCollection 2020.
[12] Yang Z, Miao R, Li G, Wu Y, Robson S C, Yang X, Zhao Y, Zhao H, Zhong Y. Identification of recurrence related microRNAs in hepatocellular carcinoma after surgical resection. Int J Mol Sci. 2013 Jan. 8; 14(1):1105-18. doi: 10.3390/ijms14011105.
[13] Lu C Y, Hsieh S Y, Lu Y J, Wu C S, Chen L C, Lo S J, Wu C T, Chou M Y, Huang T H, Chang Y S. Aberrant DNA methylation profile and frequent methylation of KLK10 and OXGR1 genes in hepatocellular carcinoma. Genes Chromosomes Cancer. 2009 Dec.; 48(12):1057-68. doi: 10.1002/gcc.20708.
[14] Leygo C, Williams M, Jin H C, Chan M W Y, Chu W K, Grusch M, Cheng Y Y. DNA Methylation as a Noninvasive Epigenetic Biomarker for the Detection of Cancer. Dis Markers. 2017; 2017:3726595. doi: 10.1155/2017/3726595. Epub 2017 Sep. 5.
[15] Luo H, Wei W, Ye Z, Zheng J, Xu R H. Liquid Biopsy of Methylation Biomarkers in Cell-Free DNA. Mol Trends Med. 2021 May; 27(5):482-500. doi: 10.1016/j.molmed.2020.12.011. Epub 2021 Jan. 23.

### SUMMARY

In the present invention, the critical issue of early recurrence after surgical resection of liver cancer from an epigenetic perspective is addressed. At first, genome-wide methylation profiles generated by a computing system were used to identify differential methylation changes between HCC patients with and without early recurrence. Potential methylation biomarkers were validated by quantitative methylation-specific PCR (qMSP), and a prediction model was established to accurately distinguish early recurrence in HCC patients.

In the present invention, a detection and quantification method measuring in a computing manner the methylation levels of genes as biomarkers for early recurrence of liver cancer is provided. The biomarker evaluation of these methylated genes will complement the conventional prediction tools and facilitate more accurate detection of early recurrence of liver cancer.

Several novel genes regulated by DNA methylation were identified in a computing system as participating in early recurrence (ER) and may potentially act as a powerful predictor for prediction and prognosis. The methylation prediction model constructed in a computing manner in the present invention has proven to be effective in predicting early recurrence (ER) and survival of HCC patients. This model exceeds the predictive capabilities of current clinicopathological factors and serum markers. When combined with other markers, the model provides even more accurate predictions for early recurrence.

This invention discloses the role of DNA methylation in predicting the recurrence and survival of HCC patients who underwent liver resection. A genome-wide approach to compare the methylation profiles of early recurrence (ER) and recurrence-free (RF) groups, both with and without cirrhosis. Early recurrence (ER) and recurrence-free (RF) groups had distinct signatures of hypermethylation and hypomethylation. Two methods, Combined Bisulfite Restriction Analysis (COBRA) and qMSP, were used to measure the methylation levels of various genes in different groups of patients, based on their recurrence status and cirrhosis condition. Some genes were differentially methylated between early recurrence (ER) and recurrence-free (RF) patients, and these genes could be used to construct “a methylation prediction model for ER (MER).” This invention compared MER with other clinical factors, such as AFP, tumor size, and BCLC staging, and found that MER had a better performance in predicting early recurrence (ER), as well as disease-free survival (DFS) and overall survival (OS). Furthermore, Cox regression was used to identify the independent prognostic factors for DFS and OS and found that MER and BCLC were the only significant ones. This invention concluded that methylation markers could improve the prognostic prediction of HCC recurrence and survival.

Various other objects, advantages, and features of the present invention will become readily apparent from the ensuing detailed description accompanying drawings, and the novel features will be particularly pointed out in the appended claims.

### BRIEF DESCRIPTION OF DRAWINGS

The following detailed descriptions, given by way of example, and not intended to limit the present invention solely thereto, will be best be understood in conjunction with the accompanying figures:

FIG. 1 illustrates CpG microarrays of hypermethylated and hypomethylated genes in HCC early recurrence using an unsupervised hierarchical clustering analysis of the data from the 24 HCC patients including 14 non-cirrhotic (cirrhosis-free) patients and 10 cirrhotic (cirrhosis) patients. Each row represents a gene, and each column represents a tissue sample. Hypermethylated and hypomethylated genes are indicated by hyper and hypo, respectively.

FIG. 2A and FIG. 2B illustrate gene ontology and pathway analysis of aberrantly methylated genes in cirrhosis-free and cirrhosis liver tissue samples, respectively. All pathways presented have statistical significance (P-value <0.05).

FIG. 3A and FIG. 3B schematically illustrate interaction networks of differentially methylated genes associated with early recurrence in non-cirrhotic (cirrhosis-free) and cirrhotic (cirrhosis) HCC patients, respectively. The network of cirrhosis-free HCC consists of 21 nodes and 45 edges and the enrichment p-value is below 1.0e⁻¹⁶. The network of cirrhotic HCC consists of 23 nodes and 24 edges and the enrichment p-value is 4.47e⁻¹¹. Each node represented all the proteins produced by a single, protein coding gene locus and each edge represented the predicted functional associations.

FIG. 4A, FIG. 4B, and FIG. 4C show differentially methylated genes in HCC patients under the COBRA analysis. FIG. 4A illustrates a representative COBRA result of CRTC1 in 6 ER and 6 RF samples from the cirrhosis-free HCC patients. FIG. 4B shows a summary of the COBRA results in cirrhosis-free HCC patients composed of 6 ER and 6 RF patients. The dark bar represents the case showing hypermethylation. A single gene may have multiple designed COBRA PCR regions each of which may be denoted by an alphabet following the gene name. FIG. 4C shows a summary of COBRA results in the cirrhotic HCC patients in which 5 ER patients and 5 RF patients were tested for hypomethylated genes, while 3 ER patients and 5 RF patients were tested for hypermethylated genes.

FIG. 5 shows methylation levels of 10 candidate genes in ER and RF groups from HCC patients, including 3 hypermethylated genes and 7 hypomethylated genes. Methylation levels were transformed by nature log and depicted by box plots. Boxes extend from 25th to 75th percentile and are divided by a solid line that represents the median of each group. Whiskers extend from the 5th to the 95th percentile. Each outlier is denoted by a dot. T test was used to determine statistical significance.

FIG. 6A to FIG. 6D illustrate receiver operator characteristic (ROC) curves for MER, AFP, BCLC, and tumor size, respectively.

FIG. 7A to FIG. 7G illustrate the ROC curves for the combination of any two, three, or all of MER, AFP, BCLC, and tumor size, respectively.

FIG. 8A to FIG. 8D illustrate the prognostic abilities of the predictors MER, AFP, tumor size, and BCLC stage for the disease-free survival (DFS), respectively, in HCC patients using Kaplan-Meier analysis. FIG. 9A to FIG. 9D illustrate the prognostic abilities of the predictors MER, AFP, tumor size, and BCLC stage for the overall survival (OS), respectively, in HCC patients using Kaplan-Meier analysis. Patients were classified as high- and low-risk groups based on the following cutoffs: MER at 0.45, AFP at 20 ng/ml, tumor size at 5 cm, and BCLC stages 0, A, B, and C.

FIG. 10A and FIG. 10B are Forest plots showing the hazard ratios (HRs) of risk factors for the disease-free survival (DFS) and the overall survival, respectively.

### DETAILED DESCRIPTION OF THE EMBODIMENTS

In the following description, the biomarker/biomarkers, and the corresponding embodiments of the detection/validation/identification/quantification methods are set forth as preferred examples. It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

In one embodiment of the present invention, a genome-wide approach is applied in a computing system, such as a computer or a system including a microprocessor capable of computing data and processing bio-signals, to first perform identification of a set of biomarkers for detecting and quantifying liver cancer from novel potential methylation biomarkers in a plurality of hepatocellular carcinoma (HCC) patients, comprising cirrhotic and cirrhosis-free patients. The cirrhotic and cirrhosis-free patients are separately subdivided into early recurrence (hereinafter ER) and recurrence-free (herein after RF) groups. The computing system is designed to process, analyze, simulate, and model biological data and may employ specialized algorithms, software, and high-performance hardware to study and solve complex problems in biology, bioinformatics, computational biology, and other life science domains. Common applications of the computing system include genome sequencing and assembly, molecular modeling and simulation, bioinformatics databases, gene expression analysis, protein structure prediction, and drug design. Key components that enable the computing system to handle big biological data include clustering algorithms, artificial intelligence/machine learning models, distributed storage and cloud computing, as well as advanced visualization capabilities. In one example, the computing system may include a biomaterial input device, a biomaterial processing unit, a bio-signal detection unit, and a data processing unit incorporating a microprocessor.

In one example, the HCC patients who participated in the biomarkers identification are 24 in number and were selected from 64 HCC patients undergoing surgical resection, who were enrolled from National Cheng Kung University (NCKU) Hospital, Taiwan. The 64 HCC patients were classified as 54 cirrhosis-free patients composed of 27 ER patients and 27 RF patients, as well as 10 cirrhotic patients, composed of 4 ER patients and 6 RF patients. The patients who experience a relapse within one year after liver resection were defined as ER patients, whereas those who remain recurrence-free for over five years were defined as RF patients. All the 64 HCC patients included in the study were diagnosed with incident HCC and received hepatic resection as their initial treatment. All experimental protocols and study methods were approved by the Institutional Review Board of Human Research of NCKU Hospital. Written informed consent was obtained from the patients who provided the specimens.

In one example, tumor tissue samples were collected promptly from surgically excised specimens obtained directly from the operating room. Tissues were frozen by liquid nitrogen immediately after resection and stored at −80° C. refrigerator until needed. Genomic DNA was extracted from tumor tissue by using the DNeasy blood and tissue kit (Qiagen). The quality and quantity of the isolated genomic DNA were analyzed with an ND-1000 spectrophotometer (Nanodrop Technologies).

In one example, Human CpG microarray was used to identify novel methylated genes involved in early recurrence after resection. 500 ng of DNA was bisulfite converted and processed on the array. The methylation level for each probe was calculated as an average value scaled from 0 (unmethylated) to 1 (completely methylated), representing the ratio of methylation at specific CpG locus. To identify differentially methylated regions, array data was processed using several filtering steps, consisting of removing CpG sites with a detection p-value ≥0.05, on the X and Y chromosomes, and containing documented single nucleotide polymorphisms (SNPs).

To explore the potential mechanisms of the genes affected by DNA methylation, significantly differential methylated genes were subjected to Gene Ontology (GO) enrichment (http://geneontology.org/) and Kyoto Encyclopedia of Genes and Genomes (KEGG) pathway analyses (https://www.kegg.jp/). In one example, the Database for Annotation, Visualization, and Integrated Discovery (DAVID), which is a comprehensive set of functional annotation tools (https://david.ncifcrf.gov/), was used. GO terms included biological process, cellular component, and molecular function. The specific cut-off used for terms and pathways was 0.05, and the top 10 of GO terms and pathways were selected.

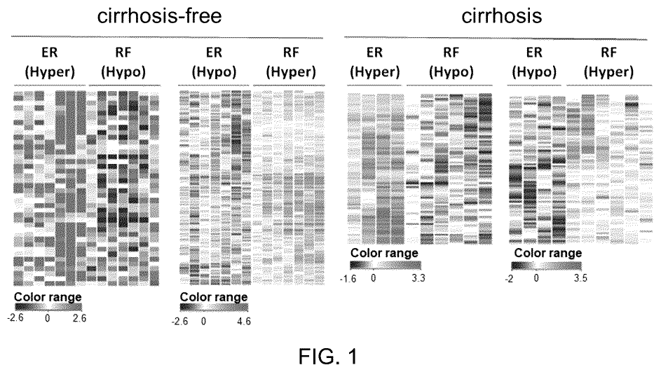

FIG. 1 illustrates CpG microarrays of hypermethylated and hypomethylated genes in HCC early recurrence using an unsupervised hierarchical clustering analysis of the data from the 24 HCC patients including 14 non-cirrhotic (cirrhosis-free) patients and 10 cirrhotic (cirrhosis) patients. Each row of the microarrays represents a gene, and each column of the microarrays represents a tissue sample. Hypermethylated and hypomethylated genes are indicated by hyper and hypo, respectively.

As shown in FIG. 1, the ER and RF groups show distinct signatures of hypermethylation and hypomethylation in both non-cirrhotic (cirrhosis-free) and cirrhotic (cirrhosis) HCC patients. For the cirrhosis-free patients, 40 significant hypermethylated regions and 297 significant hypomethylated regions of the ER group were identified. For the cirrhotic patients, 181 hypermethylated and 77 hypomethylated regions were obtained in the ER group.

Further, Search Tool for Retrieval of Interacting Genes (STRING) was used to separately analyze the 337 differential methylated regions including the above-mentioned 40 hypermethylated regions and 297 hypomethylated regions of the cirrhosis-free HCC patients, and the 258 differentially methylated regions including the above-mentioned 181 hypermethylated regions and 77 hypomethylated regions of the cirrhosis HCC patients to build up the direct interaction networks of genes regulated by DNA methylation and identify key hub genes.

In one example, the interaction network of differentially methylated genes was constructed using the STRING (https://string-db.org) database, which integrates both known and predicted protein-protein interactions (PPIs). In the networks, the nodes correspond to the proteins and the edges represent the interactions. The interactions include direct (physical) and indirect (functional) associations. Interaction sources included experimental repositories, computational prediction methods, and public text collections. The selected species was limited to Homo sapiens, and a combined score >0.4 was applied. For the network, PPI enrichment p-value and average local clustering coefficient were reported.

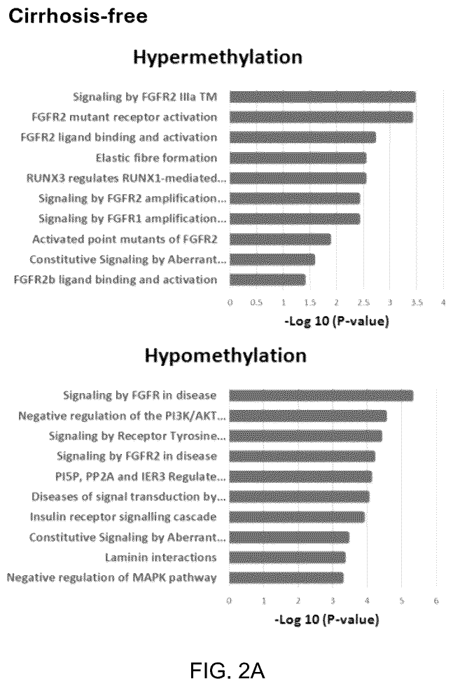

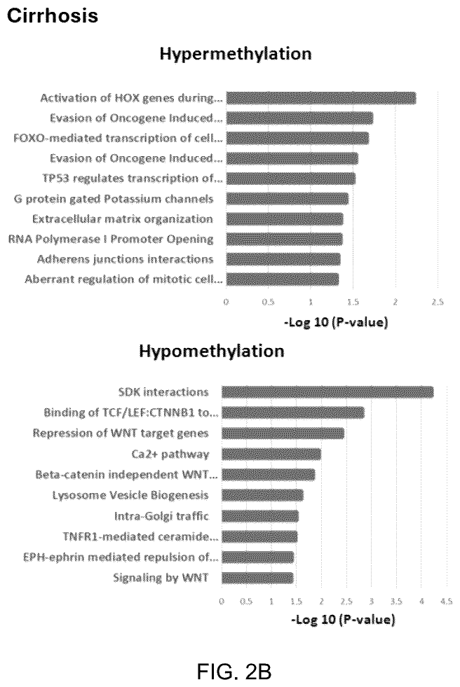

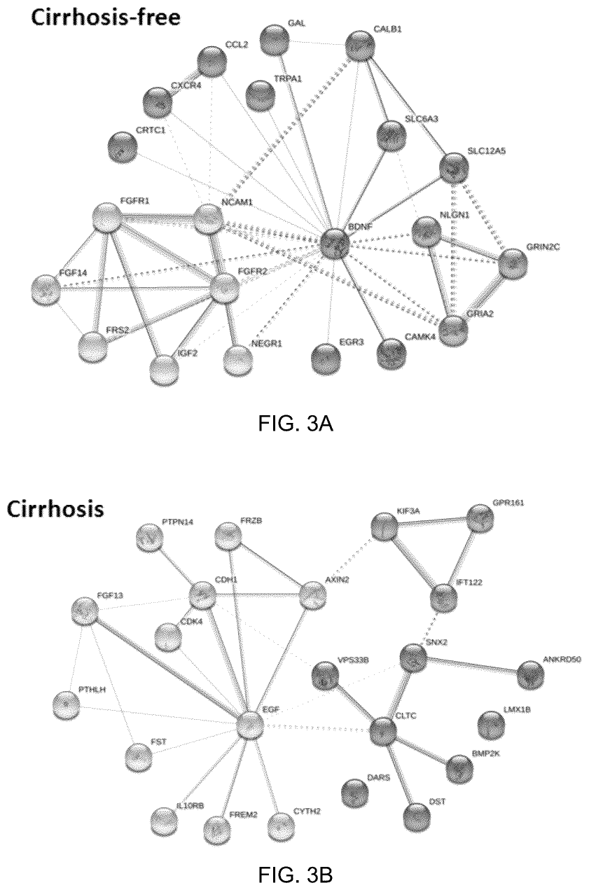

FIG. 2A and FIG. 2B illustrate gene ontology and pathway analysis of aberrantly methylated genes in cirrhosis-free and cirrhosis liver tissue samples, respectively. All pathways presented have statistical significance (p-value <0.05). As shown in FIG. 2A, the enrichment and clustering showed several hub genes including BDNF, FGFR1, and FGFR2 in the differentially methylated regions of the cirrhosis-free HCC patients. FIG. 3A and FIG. 3B schematically illustrate interaction networks of differentially methylated genes associated with early recurrence in non-cirrhotic and cirrhotic HCC patients. For the non-cirrhosis-free HCC, the network consists of 21 nodes and 45 edges, and the enrichment p-value is below 1.0e⁻¹⁶. For the cirrhotic HCC, the network consists of 23 nodes and 24 edges, and the enrichment p-value is 4.47e⁻¹¹. Each node represents all the proteins produced by a single, protein-coding gene locus, and each edge represents the predicted functional associations. As shown in FIG. 3B, EGF dominates among the differentially methylated regions of the cirrhotic HCC in the STRING analysis. These results correspond to our previous findings and strongly indicate that EGFR and Wnt signaling crosstalk transactivate one another in cancer development.

As is well known, Combined Bisulfite Restriction Analysis (COBRA) is a method of qualitative methylation assay used to validate the results obtained from the CpG microarray. In one embodiment, a plurality of HCC clinical samples from cirrhosis-free and cirrhotic HCC patients were examined with the COBRA analysis for hypermethylated and hypomethylated genes.

In one example, 22 clinical samples from cirrhosis-free HCC patients, including 6 ER samples and 6 RF samples, were examined with the COBRA analysis. On the other hand, 5 ER samples and 5 RF samples from cirrhotic HCC patients were tested for hypomethylated genes, and 3 ER samples and 5 RF samples from cirrhotic HCC patients were tested for hypermethylated genes.

In one example, Genomic DNA (1 μg) from HCC clinical samples was bisulfite-converted by treatment of sodium bisulfite. Bisulfite converted genomic DNA was subjected to polymerase chain reaction (PCR) which was performed using 1 μl of converted DNA in a 20 μl PCR reaction containing 0.5 μM of each primer and 1× PCR Master Mix. The PCR conditions were as follows: 95° C. for 3 min, 40 cycles of 95° C. for 3 sec, annealing temperature for 20 sec, 72° C. for 10 sec, and final extension at 72° C. for 20 sec. Primer and probe sequences are available upon request. Amplified DNA was digested using appropriate restriction enzymes that recognize at least one CpG site on their recognition sequences. Digested DNA fragments were visualized on 1.5% (w/v) ethidium bromide-stained agarose gels.

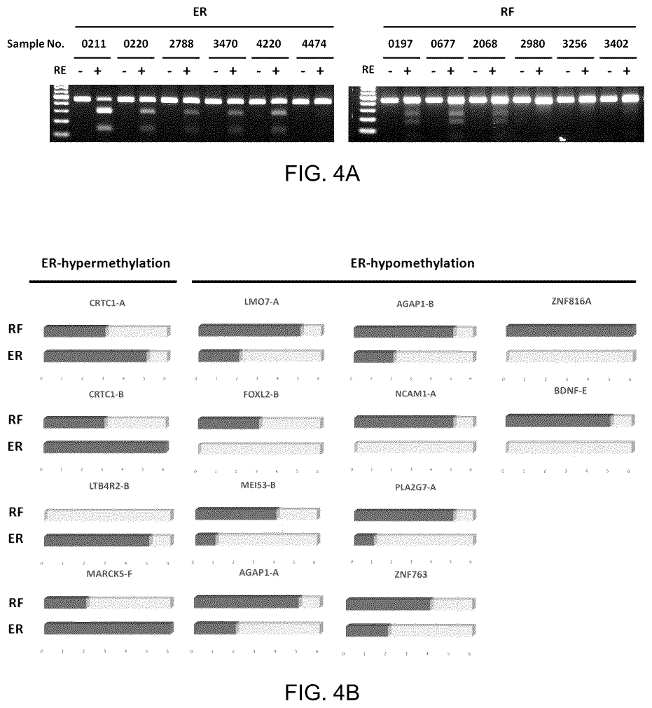

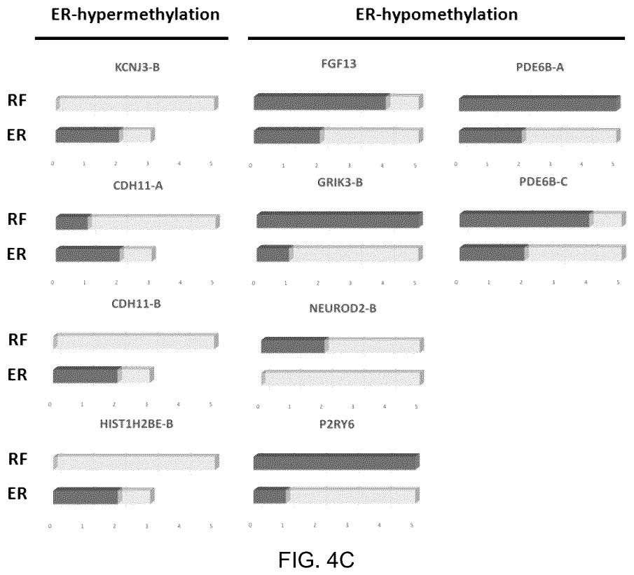

FIG. 4A, FIG. 4B, and FIG. 4C show differentially methylated genes in HCC patients under the COBRA analysis. FIG. 4A illustrates a representative COBRA result of CRTC1 in the 6 ER and 6 RF samples from the cirrhosis-free HCC patients. In FIG. 4A, the plus or the minus sign indicates that the PCR products obtained in a PCR amplification of sodium bisulfite-converted DNA were incubated with or without restriction enzyme (RE), wherein the first lane is a 100 bp marker. FIG. 4B shows a summary of the COBRA results in the 6 ER and 6 RF samples from the cirrhosis-free HCC patients, wherein the dark bar represents the case showing hypermethylation. A single gene may have multiple designed COBRA PCR regions each of which may be denoted by an alphabet following the gene name. FIG. 4C shows a summary of the COBRA results in the cirrhotic HCC patients in which 5 ER patients and 5 RF patients were tested for hypomethylated genes, while 3 ER patients and 5 RF patients were tested for hypermethylated genes.

As shown in FIG. 4A, CRTC1 hypermethylation can be observed in 5 out of the 6 ER samples from the cirrhosis-free HCC patients. In contrast, only 3 RF samples show CRTC1 hypermethylation. As shown in FIG. 4B, in the ER samples from the cirrhosis-free HCC patients, CRTC1, LTB4R2, and MARCKS show hypermethylation, while LMO7, FOXL2, MEIS3, AGAP1, BDNF, NCAM1, PLA2G7, ZNF763, and ZNF816A are found to be hypomethylated. As shown in FIG. 4C, in the ER samples from the cirrhotic HCC patients, KCNJ3, CDH11, and HISTIH2BE show hypermethylation, while FGF13, GRIK3, NEUROD2, P2RY6, and PDE6B are comparatively found to be hypomethylated.

In one embodiment, additional cirrhosis-free HCC patients were recruited to serve as a validation set to examine their methylation levels by qMSP. In one example, 40 cirrhosis-free HCC patients were recruited as the validation set, containing 20 patients each in the ER and RF groups. The 40 cirrhosis-free HCC patients were selected from the above-mentioned 64 HCC patients undergoing surgical resection, who were enrolled from National Cheng Kung University (NCKU) Hospital, Taiwan.

In one example, Bisulfite converted DNA, as described above, was amplified by real-time quantitative methylation-specific PCR (qMSP) using fluorescent probes. Each reaction involved 1× qPCR Master Mix, 0.5 μM of each primer and 0.25 μM of probe in a total volume of 20 μl. Amplification was performed on the StepOnePlus Real-Time PCR System (Thermo Fisher Scientific). The methylation level of a methylated gene may be calculated from the formula: 2^{[Ct(housekeeping gene)-Ct(biomarker)]}×100, wherein Ct is the value of single data points derived from real-time PCR amplification plots, and the housekeeping gene may be a gene selected from a group consisting of B-actin, GAPDH, HPRT, YWHAZ, ARBP, SDHA and UBC. In one example, the methylation level was calculated as the difference in Ct value between beta-actin and candidates using the following formula: 2^{[Ct(beta-actin)-Ct(candicate)]}×100. In one example, the primer-pairs and probe utilized for detecting methylation levels of the LTB4R2 gene, CRTC1 gene, MARCKS gene, MEIS3 gene, FOXL2 gene, PLA2G7 gene, LMO7 gene, BDNF gene, AGAP1 gene, and NCAM1 gene are illustrated as follows:

TABLE 1

| Target | | |
|---|---|---|
| gene | Primer/Probe | Sequence |
| LTB4R2 | Sense primer | CGTNTGTTRTTGRCGNTTT |
| | | (SEQ ID No. 1) |
| | Antisense | CCGAYGACGNATAACACNACTA |
| | primer | (SEQ ID No. 2) |
| | Probe | TGTTCGTCRTTTCGGNCGTCRTTT |
| | | (SEQ ID No. 3) |
| CRTC1 | Sense primer | TGAGRAGTCGYTATATGGATTAGATG |
| | | (SEQ ID No. 4) |
| | Antisense | ACAACTAACYTCGACCAANCA |
| | primer | (SEQ ID No. 5) |
| | Probe | ATACACYCACCACGNAYCGAACAC |
| | | (SEQ ID No. 6) |
| MARCKS | Sense primer | TAGTTCGGTAGAGAAGGAGGTT |
| | | (SEQ ID No. 7) |
| | Antisense | CGTCYTCGACCTTAWACGAAA |
| | primer | (SEQ ID No. 8) |
| | Probe | CGAGGTNGTCGAGTTCRGTTCGTT |
| | | (SEQ ID No. 9) |
| MEIS3 | Sense primer | GGGTTGRTAGGTATGTAATYGTTT |
| | | (SEQ ID No. 10) |
| | Antisense | CCAAACCNRAACAAWCGTCTA |
| | primer | (SEQ ID No. 11) |
| | Probe | CGCTTCTWAACGTNTCTACGCTYCC |
| | | (SEQ ID No. 12) |
| FOXL2 | Sense primer | GTTGGGAGATGACGAGGATTT |
| | | (SEQ ID No. 13) |
| | Antisense | AACGCCTTTYCTCGCTWAA |
| | primer | (SEQ ID No. 14) |
| | Probe | TCGGTTAARGTTTAGGAAGTTNGGGT |
| | | (SEQ ID No. 15) |
| PLA2G7 | Sense primer | AAAGGAGTYGGTGAGTACGAYAWT |
| | | (SEQ ID No. 16) |
| | Antisense | ACCAYAACYCGCACCTAAAC |
| | primer | (SEQ ID No. 17) |
| | Probe | TTTGTTCNCRGCGGGTTAANTT |
| | | (SEQ ID No. 18) |
| LMO7 | Sense primer | GGCGTTYGTTTGTCRGTAT |
| | | (SEQ ID No. 19) |
| | Antisense | AAACCNTAACCCAACTCTACAC |
| | primer | (SEQ ID No. 20) |
| | Probe | AACTACRCTACTCCTNCRAAACGC |
| | | (SEQ ID No. 21) |
| BDNF | Sense primer | GTTTGGYGGGTGARATAGAA |
| | | (SEQ ID No. 22) |
| | Antisense | TTCCRACAATTCGCTATYCC |
| | primer | (SEQ ID No. 23) |
| | Probe | CGGCGCNGAGTYGTTTAGATTTGT |
| | | (SEQ ID No. 24) |
| AGAP1 | Sense primer | GGTAGTTARAAATGATTCGGGANAGA |
| | | (SEQ ID No. 25) |
| | Antisense | CCCATWCATCACCYTCAACA |
| | primer | (SEQ ID No. 26) |
| | Probe | ACTTYAACCACCGTCTNACGACTCC |
| | | (SEQ ID No. 27) |
| NCAM1 | Sense primer | TTRATGTTCGGTAWTATTTGTGGYC |
| | | (SEQ ID No. 28) |
| | Antisense | AAAWCCTTACGCTAWCCCGRAT |
| | primer | (SEQ ID No. 29 ) |
| | Probe | AACCTYACTATATCCTWCCACCANGC |
| | | (SEQ ID No. 30) |
| β - | Sense primer | GGTGGAGGTAGTTAGGGTTTATTTGTA |
| actin | | (SEQ ID No. 31) |
| | Antisense | CCACACCACAAAATCACACTTAACCTCATTT |
| | primer | (SEQ ID No. 32) |
| | Probe | CACTTTTATTCAACTAATCTC |
| | | (SEQ ID No. 33) |

Given the above, a kit used to detect a methylation biomarker of recurrence of liver cancer can be made of a plurality of primer and probe sets of methylation biomarkers and a qPCR master mix, wherein each of the primer and probe sets may have LTB4R2 gene (SEQ ID No. 1-3 of Table 1), CRTC1 gene (SEQ ID No. 4-6 of Table 1), MARCKS gene (SEQ ID No. 7-9 of Table 1), MEIS3 gene (SEQ ID No. 10-12 of Table 1), FOXL2 gene (SEQ ID No. 13-15 of Table 1), PLA2G7 gene (SEQ ID No. 16-18 of Table 1), LMO7 gene (SEQ ID No. 19-21 of Table 1), BDNF gene (SEQ ID No. 22-24 of Table 1), AGAP1 gene (SEQ ID No. 25-27 of Table 1), and NCAM1 gene (SEQ ID No. 28-30 of Table 1), and the qPCR master mix may have Taq DNA polymerase, dNTPs, MgCl2 and buffer.

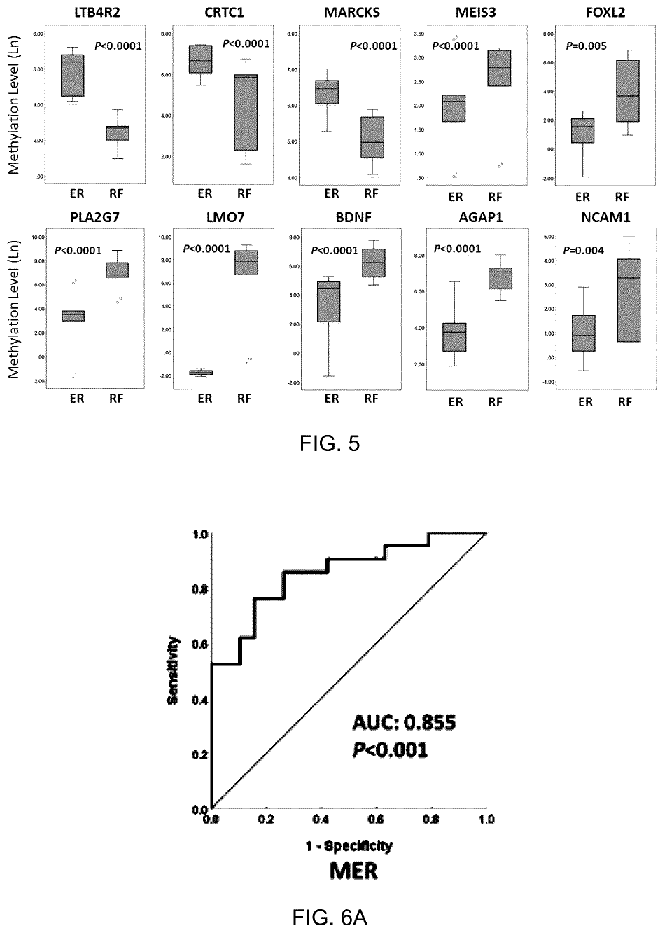

FIG. 5 shows methylation levels of 10 candidate genes in ER and RF groups from HCC patients, including 3 hypermethylated genes and 7 hypomethylated genes. Methylation levels were transformed by nature log and depicted by box plots. Boxes extend from the 25th to the 75th percentile and are divided by a solid line that represents the median of each group, whiskers extend from the 5th to the 95th percentile, and each outlier is denoted by a dot. An independent sample t-test was used to determine the difference of methylation levels in ER and RF patients.

Furthermore, logistic regression analysis was used to generate a methylation prediction model with differentially methylated genes, including BDNF, FOXL2, LMO7, NCAM1, NEIS3, PLA2G7, and LTB4R. In one example, the methylation prediction model generated by the logistic regression analysis may be represented by a formula as follows. MER=−0.941-X1×A1+X2×A2+X3×A3+X4×A4+X5×A5+X6×A6−X7×A7, wherein X1 ranges from −0.007 to 0.043, X2 ranges from −0.019 to 0.025, X3 ranges from −0.007 to 0.074, X4 ranges from −0.046 to 0.079, X5 ranges from 0.001 to 0.159, X6 ranges from −0.002 to 0.006, X7 ranges from −0.026 to 0.006, and A1, A2, A3, A4, A5, A6, and A7 represent methylation levels of BDNF, FOXL2, LMO7, NCAM1, MEIS3, PLA2G7, and LTB4R2, respectively. In the present invention, a prediction of the risk of early recurrence in liver cancer is based on the weights of the methylation levels of the biomarkers selected in the above-mentioned methylation prediction model.

In one embodiment, a receiver operating characteristic (ROC) curve analysis was used to assess the performance or the diagnostic effect of MER, i.e., the methylation prediction model for ER. Specifically, the area under the curve, cutoff value, sensitivity, and specificity were estimated.

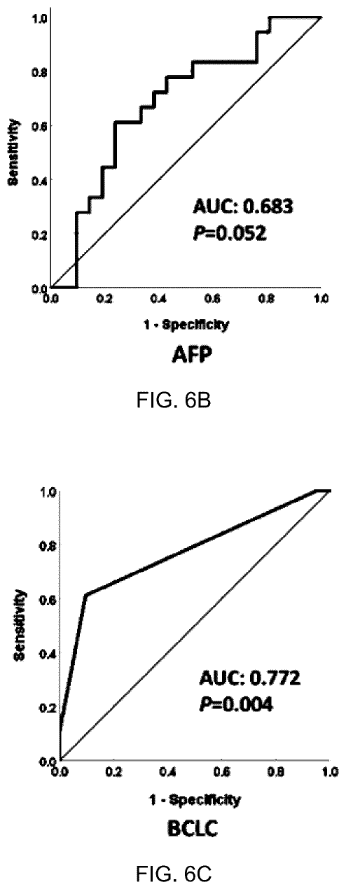

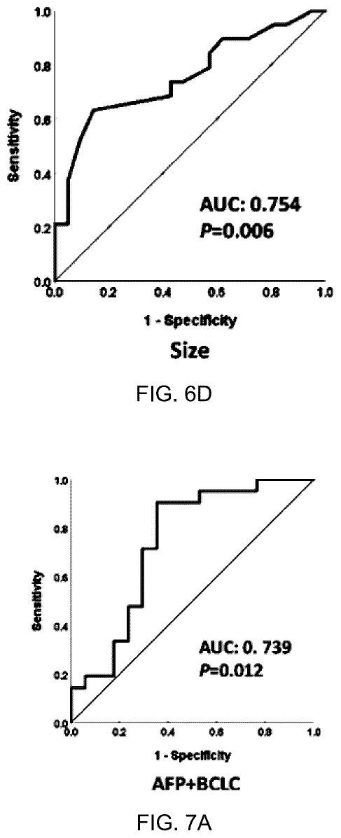

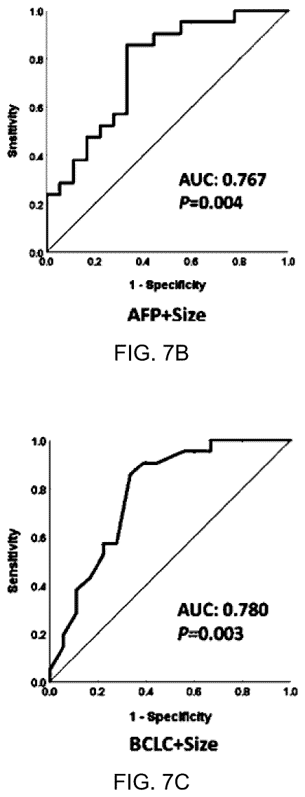

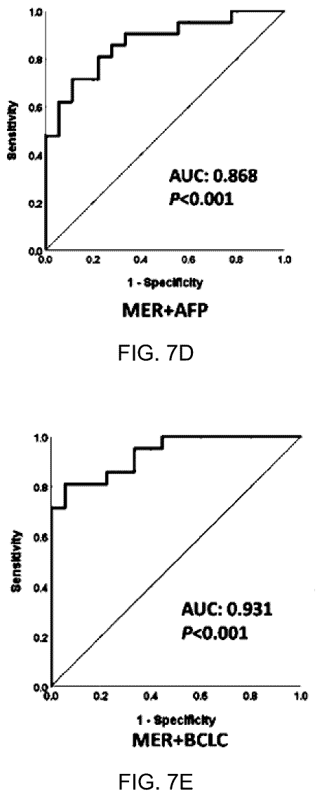

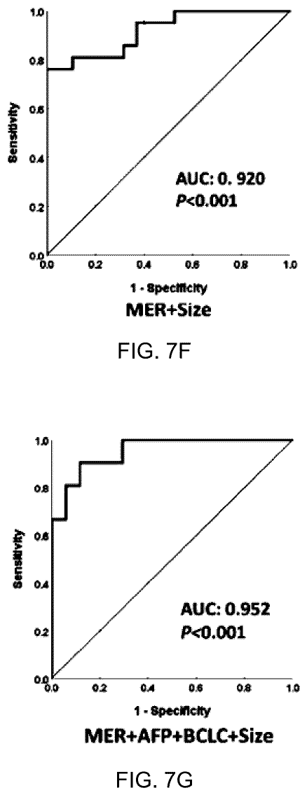

FIG. 6A to FIG. 6D illustrate receiver operator characteristic (ROC) curves for MER, AFP, BCLC, and tumor size, respectively. FIG. 7A to FIG. 7G illustrate the ROC curves for the combination of any two, three, or all of MER, AFP, BCLC, and tumor size, respectively.

As shown in FIG. 6A to FIG. 6D, the AUC (area under the curve) in view of the methylation prediction model for ER (MER) is 0.855 (95% CI: 0.738-0.971, P<0.001). The AUCs in view of AFP, BCLC, and tumor size alone are 0.683 (95% CI: 0.511-0.854, P=0.052), 0.772 (95% CI: 0.619-0.926, P=0.004), and 0.754 (95% CI: 0.600-0.909, P=0.006), respectively. As shown in FIG. 7A to FIG. 7G, in cases where any two current predictors are combined, the AUC ranges from 0.739 (95% CI: 0.569-0.910, P=0.012) in view of AFP and BCLC to 0.780 (95% CI: 0.629-0.932, P=0.003) in view of tumor size and BCLC. In cases where MER is integrated with AFP, tumor size or BCLC, the AUCs are dramatically elevated to 0.868 (95% CI: 0.756-0.979, P<0.001), 0.920 (95% CI: 0.838-1.000, P<0.001) or 0.931 (95% CI: 0.857-1.000, P<0.001), respectively. In cases where MER is combined with all three current predictors, the AUC achieves a maximum value of 0.952. (95% CI: 0.893-1.000, P<0.001).

The receiver operating characteristic (ROC) analysis also determines that the sensitivity and specificity are 85.7% and 73.7% for MER, 73.7% and 57.1% for tumor size, 72.2% and 61.9% for AFP, and 61.1% and 90.5% for BCLC, respectively. The combinations of MER and AFP, MER and tumor size, MER and BCLC have sensitivity/specificity of 85.7%/72.7%, 81.0%/89.5%, and 81.0%/94.4%, respectively. When MER is combined with all three current predictors, the resulting sensitivity and specificity are 90.5% and 88.2%, respectively. These results indicated that the performance of MER is superior to any current predictors alone or even in any combination. Integration of MER and all current predictors provides more precise predictive ability for ER patients.

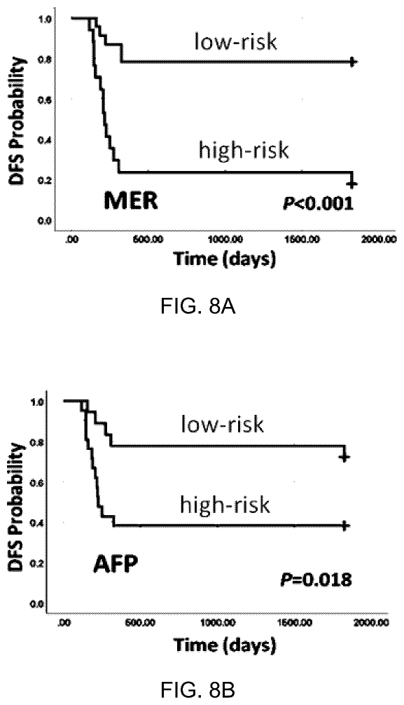

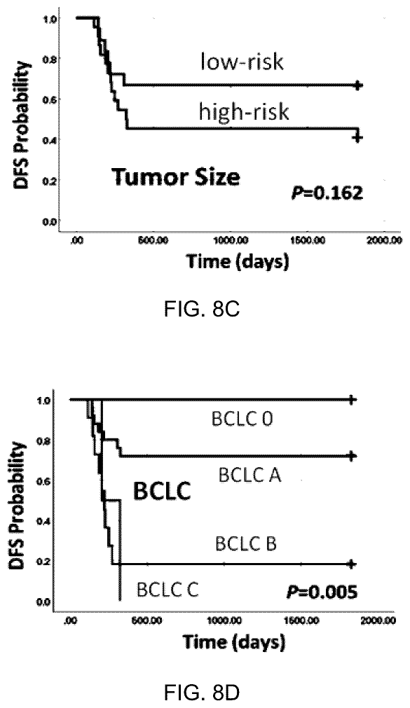

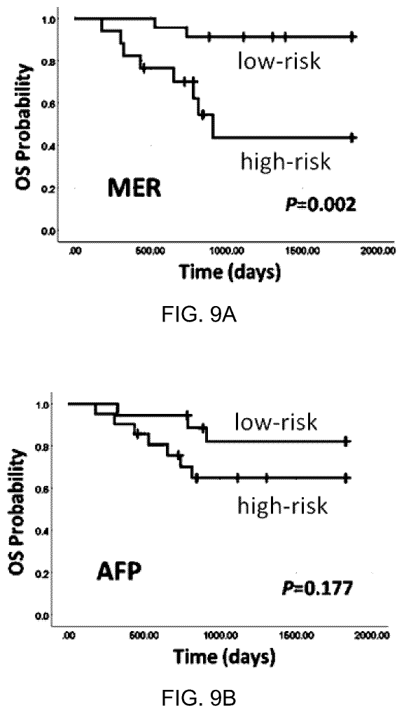

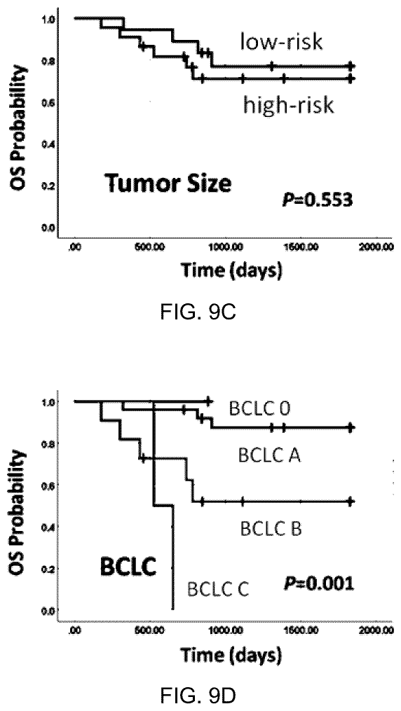

Considering that the predictors for early recurrence are highly related to clinical outcomes, we examined the prognostic abilities of these predictors for survival. In one embodiment, patients were divided into high and low risk groups based on MER, AFP, tumor size, and BCLC, and the Kaplan-Meier method was used to analyze disease-free survival (DFS) and overall survival (OS) of the patients. Specifically, survival curves were calculated by the Kaplan-Meier method and distributions were compared using the log-rank test. Disease-free survival was calculated from the date of diagnosis until recurrence or end of follow-up, and overall survival was calculated from the date of diagnosis until disease-caused death or end of follow-up. FIG. 8A to FIG. 8D illustrate the prognostic abilities of the predictors MER, AFP, tumor size, and BCLC stage for disease-free survival (DFS), respectively, in HCC patients using Kaplan-Meier analysis. FIG. 9A to FIG. 9D illustrate the prognostic abilities of the predictors MER, AFP, tumor size, and BCLC stage for overall survival (OS), respectively, in HCC patients using Kaplan-Meier analysis. Patients were classified as high- and low-risk groups based on the following cutoffs: MER at 0.45, AFP at 20 ng/ml, tumor size at 5 cm, and BCLC stages 0, A, B, and C.

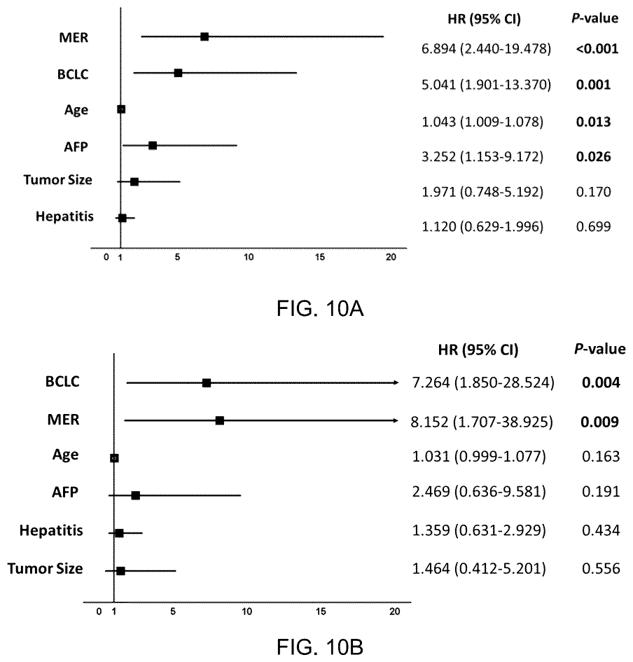

Additionally, a Cox proportional hazards regression model was applied to assess the association between each risk factor and survival. The Cox proportional hazards regression model was used in multivariate analyses and used to estimate Hazard Ratios (HRs) and their 95% confidence intervals (CIs). Statistical significance was accepted when P<0.05 for all tests. FIG. 10A and FIG. 10B are Forest plots showing the hazard ratios (HRs) of risk factors for the disease-free survival (DFS) and the overall survival, respectively. The hazard ratios (HRs) and 95% confidence intervals (CIs) are obtained according to each risk factor that was estimated by the Cox proportional hazards model.

As shown in FIG. 8A to FIG. 8D, in the DFS analysis, the 5-year DFS rates of the low-risk group of the HCC patients and the high-risk group of the HCC patients were 78.3% (or 4.1 years) and 17.6% (or 1.6 years) with P<0.001 in view of MER, respectively. In view of the AFP, the low-risk group of the HCC patients also show significantly better DFS rates than the high-risk group of the HCC patients (72.2% vs. 38.1%, P=0.018). The mean DFS rates were 4.0 years and 2.2 years, respectively. Comparatively, tumor size is not significant to the DFS rates (low-risk group vs. high-risk group, 66.7%. vs. 40.9%, P=0.162). As shown in FIG. 9A to FIG. 9D, in the OS analysis, only MER is significant. The 5-year OS rates of the low-risk group of the patients and the high-risk group of the patients were 91.3% (or 4.7 years) and 43.6% (or 3.1 years) with P=0.002 in view of MER, respectively. Not surprisingly, BCLC staging was significant in both DFS and OS. These results indicate that MER is a robust tool for predicting DFS rates and OS rates for the HCC patients undergoing liver resection.

Compared with current clinicopathological factors, the Cox proportional hazards regression model determined the association of MER and the survival rate. As shown in FIG. 10A, in terms of DFS, MER (HR=6.894; 95% CI 2.440-19.478; P<0.001), BCLC (HR=5.041; 95% CI 1.901-13.370; P=0.001), age (HR=1.043; 95% CI 1.009-1.078; P=0.013), and AFP (HR=3.252; 95% CI 1.153-9.172; P=0.026) were independent prognostic factors. As shown in FIG. 10B, in terms of OS, only BCLC (HR=7.264; 95% CI 1.850-28.524; P=0.004) and MER (HR=8.152; 95% CI 1.707-38.925; P=0.009) were identified as independent predictors. These findings demonstrate that MER is a remarkably promising predictor, exhibiting superior prognostic ability for both DFS and OS, compared to current clinicopathological factors and serum markers.

This invention addresses the critical issue of early recurrence after surgical resection of liver cancer from an epigenetic perspective. A detection and quantification method was developed to computationally measure methylation levels of genes as potential biomarkers for early HCC recurrence. These methylation biomarkers were validated by quantitative methylation-specific PCR (qMSP), and a prediction model was established that can accurately distinguish HCC patients at risk for early recurrence. Evaluation of these methylated gene biomarkers complements conventional prediction tools and facilitates more accurate detection of early recurrence of liver cancer. Overall, the methylation markers identified could enhance the prognostic predictions of HCC recurrence risk and survival when incorporated into clinical decision making.

Having described at least one of the embodiments of the claimed invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure covers modifications and variations of this disclosure provided they come within the scope of the appended claims and their equivalents. Specifically, one or more limitations recited throughout the specification can be combined in any level of details to the extent they are described to accomplish the touch display devices.

What is claimed is:

1. A method of detecting early recurrence of liver cancer, comprising:
performing biomarker identification of a plurality of differentially methylated genes in a computing system designed to process, analyze, simulate, and model biological data and equipped with a microprocessor, the methylated gens being selected from a group consisting of CRTC1, LTB4R2, MARCKS, KCNJ3, CDH11, HIST1H2BE, LMO7, FOXL2, MEIS3, AGAP1, BDNF, NCAM1, PLA2G7, ZNF763, ZNF816A, FGF13, GRIK3, NEUROD2, P2RY6, and PDE6B;
performing qualitative measurement of methylation levels of the methylated genes with Combined Bisulfite Restriction Analysis (COBRA) analysis and performing quantitative measurement of the methylation levels of the methylated genes with quantitative methylation-specific PCR (qMSP) in the computing system;
performing construction of a methylation prediction model of the methylated genes based on the measured methylation levels of the methylated genes with a logistic regression analysis in the computing system; and
performing detection of early recurrence of liver cancer with the methylation prediction model in the computing system to predict the early recurrence of liver cancer.

2. The method of claim 1, wherein the step of performing biomarker identification of the methylated genes in the computing system comprises using a genome-wide approach, a CpG microarray analysis, and a Search Tool for Retrieval of Interacting Genes (STRING) analysis in the computing system.

3. The method of claim 1, wherein the step of performing the quantitative measurement of the methylation levels of the methylated genes with the quantitative methylation-specific PCR (qMSP) in the computing system comprises performing a calculation of a difference in Ct value between housekeeping gene and each of the methylated genes in the computing system and the formula 2^{[Ct(housekeeping gene)-Ct(biomarker)]}×100 is used to perform the calculation.

4. The method of claim 3, wherein the housekeeping gene is a gene selected from a group consisting of B-actin, GAPDH, HPRT, YWHAZ, ARBP, SDHA and UBC.

5. The method of claim 1, wherein the step of performing the quantitative measurement of the methylation levels of the methylated genes with the quantitative methylation-specific PCR (qMSP) in the computing system comprises using a kit to detect, the kit comprising:
a plurality of primer and probe sets targeting LTB4R2, CRTC1, MARCKS, MEIS3, FOXL2, PLA2G7, LMO7, BDNF, AGAP1, and NCAM1 genes; and
a qPCR master mix having Taq DNA polymerase, dNTPs, MgCl2 and buffer.

6. The method of claim 5, wherein each of the primer and probe sets includes a sense primer, an antisense primer and a probe separately has a sequence correspondingly associated with the targeted gene.

7. The method of claim 1, wherein the methylation prediction model is represented as MER and satisfies MER=−0.941−X1×A1+X2×A2+X3×A3+X4×A4+X5×A5+X6×A6−X7×A7, wherein X1 ranges from −0.007 to 0.043, X2 ranges from −0.019 to 0.025, X3 ranges from −0.007 to 0.074, X4 ranges from −0.046 to 0.079, X5 ranges from 0.001 to 0.159, X6 ranges from −0.002 to 0.006, X7 ranges from −0.026 to 0.006, and A1, A2, A3, A4, A5, A6, and A7 represent the methylation levels of BDNF, FOXL2, LMO7, NCAM1, MEIS3, PLA2G7, and LTB4R2, respectively.

8. The method of claim 1, further comprising performing comparison of receiver operating characteristic (ROC) curves of the methylation prediction model, AFP, BCLC, and tumor size to assess the performance of the methylation prediction model in the computing system.

9. The method of claim 1, further comprising performing evaluation of prognosis of liver cancer with the methylation prediction model in the computing system to increase liver cancer survival rate.

10. The method of claim 9, wherein the step of performing evaluation of prognosis of liver cancer with the methylation prediction model in the computing system comprises determining prognostic ability of the methylation prediction model by analyzing disease-free survival (DFS) and overall survival using Kaplan-Meier method.

11. The method of claim 9, wherein the step of performing evaluation of prognosis of liver cancer with the methylation prediction model in the computing system comprises determining association of the methylation prediction model and survival rate of the liver cancer by applying a Cox proportional hazards regression model to estimate Hazard Ratios (HRs) of the methylation prediction model for disease-free survival (DFS) and overall survival and 95% confidence intervals (CIs) of the Hazard Ratios (HRs).
